(19) United States
(12) Patent Application Publication
(10) Pub. No.: US 2018/0146928 A1
(43) Pub. Date: May 31, 2018

(54) MEASUREMENT DATA PROCESSING SYSTEM

(71) Applicant: DEUTSCHE TELEKOM AG, Bonn (DE)

(72) Inventor: Astrid BÖGER, Bad Saarow (DE)

(21) Appl. No.: 15/577,497

(22) PCT Filed: May 17, 2016
(86) PCT No.: PCT/EP2016/060982

§ 371 Date: Nov. 28, 2017

(30) Foreign Application Priority Data
May 28, 2015 (DE) 10 2015 108 447.3

(51) Int. Cl. A61B 5/00; G16H 50/20; G16H 20/30; A61B 5/0205

(52) U.S. Cl. CPC A61B 5/7221; G16H 50/20; A61B 5/021; A61B 5/0205; A61B 5/6802; G16H 20/30

(57) ABSTRACT

A measurement data processing system is provided to offer more efficient use of measurement data, particularly vital data, captured using different appliances in order to allow more accurate analysis of a state on the basis of the measurement data. A measurement data server has a measurement data capture interface configured to receive first measurement data and first calibration information from a first measurement data capture device and receives second measurement data and second calibration information from a second measurement data capture device. The measurement data server is configured to provide the first measurement data and the second measurement data by pointing to first and second calibration information of the applicable measurement data capture devices.

The present invention relates to a measurement data processing system and a method for measurement data processing, particularly for measurement data processing for measurement data for medical appliances that are worn on a body and the data of which are merged in a “health cloud” (cloud system for processing health data), particularly medical appliances that can be used for medical care, including diagnosis, therapy and nursing.

In health cloud systems today, medical appliances are used for capturing vital data, i.e. measurement data that demonstrate a state of health/sickness of the patient. Such medical appliances have a high level of accuracy in measurement data capture and are often certified to be used in a medical setting for capturing vital data, such as in the health cloud, for example.

Besides the specially certified medical appliances, there are now more and more “fitness appliances” or “wellness appliances” in the form of consumer products for the private sector being used for measuring vital data. These consumer products often have a lower level of accuracy than the certified medical measuring instruments and do not meet the standards that are prescribed for the medical sector, for example pursuant to the Medical Devices Act or the applicable European guidelines.

There is a need for the doctor and the nursing staff to base a diagnosis for the patient on the most extensive information base possible, i.e. not to use only the data of the medical appliances for the diagnosis, but rather to be able to also include measurement data produced by patients from the private setting in the diagnosis.

It is the object of the present invention to provide a concept for more efficient use of measurement data, particularly vital data, captured using different appliances in order to allow more accurate analysis of a state on the basis of the measurement data.

This object is achieved by the features of the independent claims. Advantageous development forms are the subject matter of the dependent claims.

The methods and systems presented below can be used in the cloud, for example the health cloud. In this case, cloud or cloud computing is intended to be understood to mean the approach of making available abstracted IT infrastructures, such as computation capacity, data storage, network capacities, ready software or, in this case, the server, for example, over a network in a manner dynamically matched to need. The abstracted IT infrastructure made available is also referred to as a cloud. “Health Cloud” denotes a data processing system for processing data from substantially medical appliances that meet particular demands on medical data recording and data processing.

The methods and systems presented below may be of different type. The individual elements described can be realized by hardware components or software components, for example electronic components, which can be produced using different technologies and comprise, for example, semiconductor chips, ASICs, microprocessors, digital signal processors, integrated electrical circuits, electro-optical circuits and/or passive devices.

The methods and systems presented below can be used for communicating in client-server systems or may be based on a client-server architecture. The client-server architecture is the standard concept for the distribution of tasks within a network. Tasks are distributed to different computers by means of servers and can be requested by multiple clients to accomplish their own tasks or parts thereof when needed. The tasks may be standard tasks, such as sending e-mail, receiving e-mail, web access, etc., for example, or may be specific tasks of a piece of software or of a program. A task is referred to as a service in the client-server model.

A server is a program that provides a service. Within the context of the client-server concept, another program, the client, can use this service. The communication between client and server is dependent on the service, i.e. the service determines which data are interchanged between the two. The server is on standby to be able to react to the making of contact by a client at any time. In contrast to the client, which actively requests a service, the server behaves passively and awaits requests. The rules of the communication for a service, such as the format, calling the server and the significance of the data interchanged between server and client, for example, are stipulated by a protocol specific to the respective service.

The measurement data processing system presented below may be based on the client-server concept. By way of example, the measurement data server presented below may be a server according to the client-server architecture described above and can provide its services as a server. The first and second measurement data capture devices presented below may be e.g. clients according to the client-server architecture described above and can provide their services as clients.

The communication interfaces presented below, i.e. measurement data transmission interfaces and measurement data capture interfaces between client and server, may be based on wireless networks, for example using WLAN, WiFi, Bluetooth, infrared or other short-range communication standards. The communication interfaces may also be based on wired networks, for example using Ethernet, USB, cable, etc. The protocols used for data transmission can be Voice-over-IP (VoIP) by means of IPv4 or IPv6, for example. The communication channels between the interfaces can be set up by means of the public network, for example by the Internet, a telephone network of a telephone operator, e.g. a wired network, such as a POTS, ISDN, DSL or cable network, for example, or a wireless network, such as a mobile radio network of a mobile radio operator, for example, e.g. a cellular network, for example using a mobile radio standard such as e.g. LTE, UMTS, GSM, etc. The protocols used for data or voice transmission via the communication network can be Voice-over-IP by means of IPv4 or IPv6, or ATM, STM or other long-range communication standards.

The measurement data processing system presented below can capture and process measurement data having a particular error tolerance. In this case, error tolerance is understood to mean an accuracy or quality of the measurement. The error tolerance in this context is a discrepancy between the captured measured value and the exact measured value, which for the most part is indicated as a ratio of captured measured value to exact measured value in percent or per thousand. A low error tolerance is thus linked to an accurate measured value, whereas a high error tolerance reflects a less accurate measured value.

The measurement data processing system presented below can have measurement data capture devices that are calibrated according to a legal directive, such as the Medical Devices Act, for example. The Medical Devices Act regulates particularly the requirements for marketing and commissioning medical devices. The Medical Devices Act includes a series of national directives, for example for performing monitoring of and for operating and using medical devices. Medical devices are products intended for medical use that the manufacturer intends to be used for human beings. These include, by way of example, implants, products for injection, infusion, transfusion and dialysis, software, pacemakers, x-ray equipment, medical instruments, and also laboratory diagnostic agents and in vitro diagnosis agents. The Medical Devices Act and the associated medical devices decree have been used to implement European guidelines 90/385/EEC, 93/42/EEC and 98/79/EC in national law. The purpose of this Act is to regulate dealings with medical devices and hence to ensure the safety, suitability and performance of the medical devices and also the health and necessary protection of patients, users and third parties.

According to a first aspect, the invention relates to a measurement data processing system, comprising: a measurement data server for providing first measurement data having a first error tolerance and second measurement data having a second error tolerance, the first error tolerance being lower than the second error tolerance; a first measurement data capture device for capturing the first measurement data, wherein the first measurement data capture device is calibrated for capturing the first measurement data having a first error tolerance and wherein the first measurement data capture device has a first measurement data transmission interface that is configured to transmit the first measurement data and a first piece of calibration information about the calibration of the first measurement data capture device to the measurement data server; and a second measurement data capture device for capturing the second measurement data, wherein the second measurement data capture device is calibrated for capturing the second measurement data having a second error tolerance and wherein the second measurement data capture device has a second measurement data transmission interface that is configured to transmit the second measurement data and a second piece of calibration information about the calibration of the second measurement data capture device to the measurement data server, wherein the measurement data server has a measurement data capture interface that is configured to receive the first measurement data and the first calibration information from the first measurement data capture device and to receive the second measurement data and the second calibration information from the second measurement data capture device, wherein the measurement data server is further configured to provide the first measurement data and the second measurement data by pointing to the first calibration information and the second calibration information of the applicable measurement data capture devices.

This affords the advantage that the doctor or the nursing staff can base a diagnosis of the patient on a very extensive information base, because they can use not only the data of the medical appliances alone, i.e. the very accurate measurement data of the first measurement data capture device with the low first error tolerance, for the diagnosis, but can also include measurement data produced by patients from the private setting, i.e. the less accurate measurement data of the second measurement data capture device with the higher second error tolerance, in the diagnosis as well.

It is thus possible for the medical appliances on hand and the appliances from the private setting to be used efficiently, and they thus allow more accurate analysis of the state of the patient than would be possible without additional incorporation of the second measurement data.

In one embodiment of the measurement data processing system, the first calibration information comprises a piece of information about a quality of the first measurement data and the second calibration information comprises a piece of information about a quality of the second measurement data.

This affords the advantage that the doctor or the nursing staff, during the diagnosis using the measurement data, is/are very conscious of what accuracy his/their diagnosis has, since he has/they have the information available about the quality of the measurement data.

In one embodiment of the measurement data processing system, the first measurement data capture device is a medical appliance for capturing vital data having a prescribed first error tolerance.

This affords the advantage that the measurement data processing system can be used flexibly in infrastructure on hand. If the first measurement data capture device is a medical appliance, as is on hand in hospitals, for example, then the measurement data processing system can flexibly incorporate this medical appliance into the measurement data capture. The measurement data processing system can therefore be used in the hospital in order to extend the information base on hand and provide better diagnosis options.

In one embodiment of the measurement data processing system, the first measurement data capture device has a compliance with a medical standard, particularly a compliance pursuant to the Medical Devices Act.

This affords the advantage that the first measurement data capture device meets the legal directives and can be used in a medical setting.

In one embodiment of the measurement data processing system, the first measurement data capture device has a gauging stamp and the first calibration information is based on the gauging stamp.

This affords the advantage that the first measurement data come from a gauged first measurement data capture device and hence are very accurate. On the basis of the gauging stamp, the user can be informed about the accuracy and the setting in which the first measurement data capture device can be used.

In one embodiment of the measurement data processing system, the second measurement data capture device is one of the following appliances for capturing vital data: a fitness appliance, a wellness appliance, a consumer product for home use, particularly a heart rate monitor, a pedometer, a blood pressure gauge or a wearable appliance.

This affords the advantage that the second measurement data capture device can deliver further virtual data that can round off the overall impression of the doctor or nursing staff. This increases the relevance of the measurement data obtained from the measurement data processing system.

In one embodiment of the measurement data processing system, the second measurement data capture device has an identifier, particularly a serial number, that indicates a design of the second measurement data capture device, the second calibration information being based on the identifier.

This affords the advantage that the identifier or the design of the second measurement data capture device can be used to infer a quality of the second measurement data that have been recorded by the second measurement data capture device. Hence, automated measurement data processing is also possible in which a quality of the measurement data can be automatically evaluated and displayed on the basis of the identifier or design, for example using signal colors of a set of traffic lights in red, yellow and green.

In one embodiment of the measurement data processing system, the measurement data server is designed to store the received first measurement data in a first database and to store the received second measurement data in a second database, wherein the second database is separate from the first database and wherein the first measurement data stored in the first database have a pointer to the second measurement data stored in the second database.

This affords the advantage that the first, i.e. very accurate, measurement data are stored in a first database that satisfies the legal directives, for example, and that the second, less accurate, measurement data can be stored in a separate database that does not have to satisfy the legal directives. This means that it is easier to keep the different measurement data apart, and the measurement data cannot be mistakenly mixed up or confused. The risk of an incorrect diagnosis is thereby reduced.

In one embodiment of the measurement data processing system, the first measurement data stored in the first database are provided with a reference that refers to the second measurement data stored in the second database.

This affords the advantage that the measurement data processing system can also incorporate the second measurement data in automated fashion on the basis of the reference so as to have all of the measurement data ready. Incorporation or nonincorporation can be effected optionally at the request of the doctor or nurse.

In one embodiment of the measurement data processing system, the reference comprises the second calibration information of the second measurement data capture device.

This affords the advantage that the doctor or nurse, when deciding to also incorporate further measurement data in the diagnosis, is informed about the calibration with which the second measurement data capture device is calibrated. This means that he can flexibly decide whether or not this accuracy of the calibration is adequate for his case.

In one embodiment of the measurement data processing system, the reference comprises a form factor that indicates a Q-factor of the second measurement data in relation to a Q-factor of the first measurement data.

This affords the advantage that the form factor provides a clear and rapid decision aid about the accuracy with which the second measurement data have been recorded.

According to a second aspect, the invention relates to a method for measurement data processing, having the following steps: capture of first measurement data by a first measurement data capture device, wherein the first measurement data capture device is calibrated for capturing the first measurement data having a first error tolerance, and transmission of the first measurement data and a first piece of calibration information about the calibration of the first measurement data capture device; capture of second measurement data by a second measurement data capture device, wherein the second measurement data capture device is calibrated for capturing the second measurement data having a second error tolerance, the first error tolerance being lower than the second error tolerance, and transmission of the second measurement data and a second piece of calibration information about the calibration of the second measurement data capture device; reception of the first measurement data and the first calibration information from the first measurement data capture device and reception of the second measurement data and the second calibration information from the second measurement data capture device; and provision of the first measurement data and the second measurement data by pointing to the first calibration information and the second calibration information of the applicable measurement data capture devices.

This affords the advantage that the doctor or the nursing staff can use this method to base a diagnosis of the patient on a very extensive information base, because they can use not only the data of the medical appliances alone, i.e. the very accurate measurement data of the first measurement data capture device with the low first error tolerance, for the diagnosis, but can also include measurement data produced by patients from the private setting, i.e. the less accurate measurement data of the second measurement data capture device with the higher second error tolerance, in the diagnosis as well.

It is thus possible for the medical appliances on hand and the appliances from the private setting to be used efficiently, and they thus allow more accurate analysis of the state of the patient than would be possible without additional incorporation of the second measurement data.

In one embodiment of the method, the first calibration information comprises a piece of information about a quality of the first measurement data, and the second calibration information comprises a piece of information about a quality of the second measurement data.

This affords the advantage that the doctor or the nursing staff, during the diagnosis using the measurement data, is/are very conscious of what accuracy his/their diagnosis has, since he has/they have the information available about the quality of the measurement data.

In one embodiment, the method further comprises display of the second calibration information in respect of a compliance with a medical standard, particularly in respect of a compliance pursuant to the Medical Devices Act.

This affords the advantage that the first measurement data capture device meets the legal directives and can be used in a medical setting.

According to a third aspect, the invention relates to a computer program having a program code for performing the method according to the second aspect of the invention when the computer program is executed on a computer.

This affords the advantage that the method can be used flexibly on computers on hand and can be matched to changed circumstances with an update, for example a software update.

Further exemplary embodiments are explained with reference to the accompanying drawings, in which

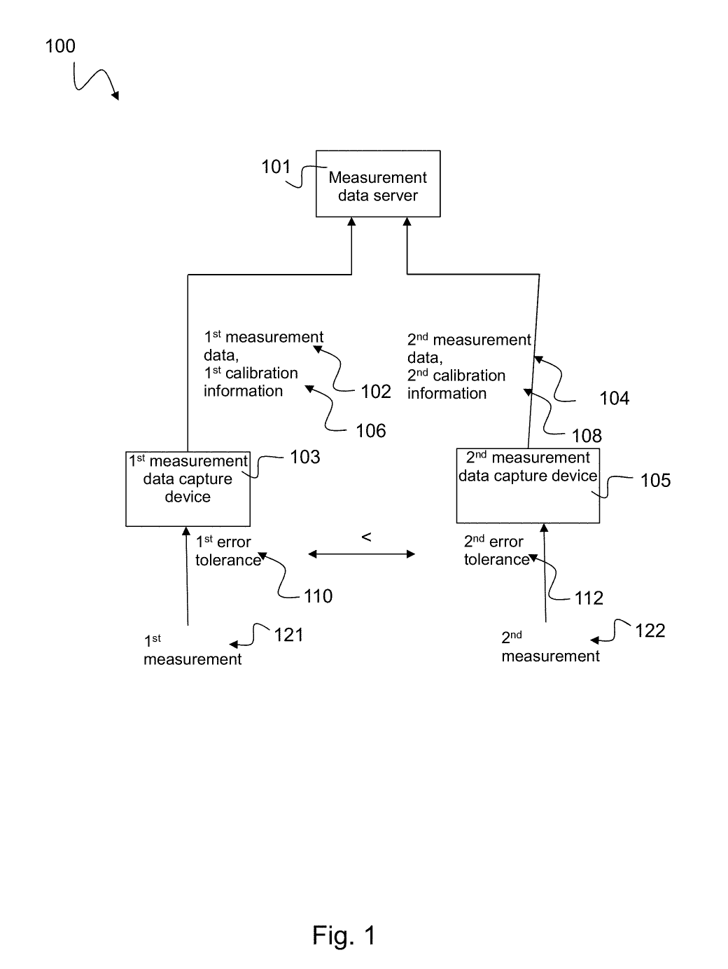

FIG. 1 shows a schematic depiction of a measurement data processing system 100 according to one embodiment;

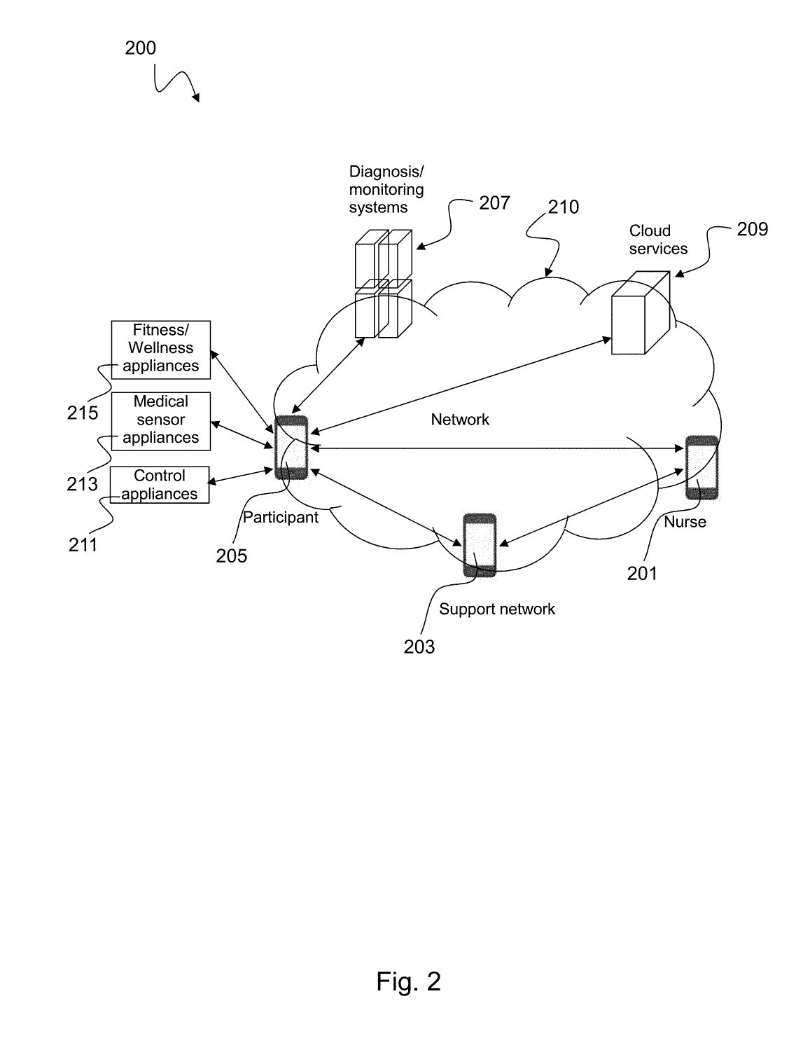

FIG. 2 shows a schematic depiction of a decentralized system 200 for processing vital data according to one embodiment;

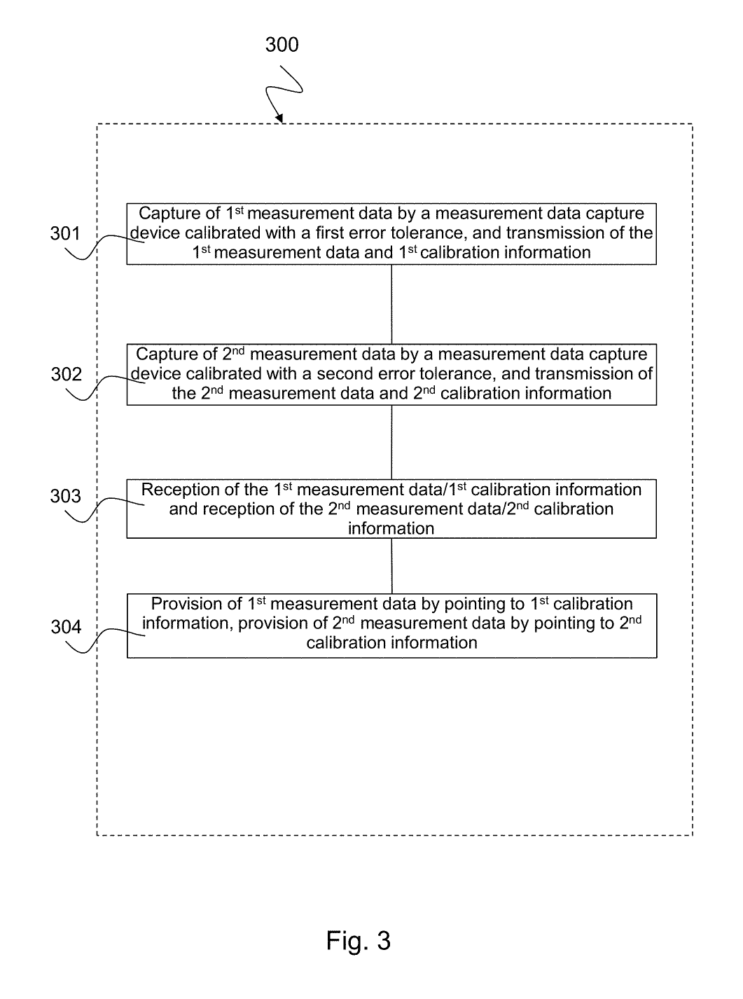

FIG. 3 shows a schematic depiction of a method 300 for measurement data processing according to one embodiment; and

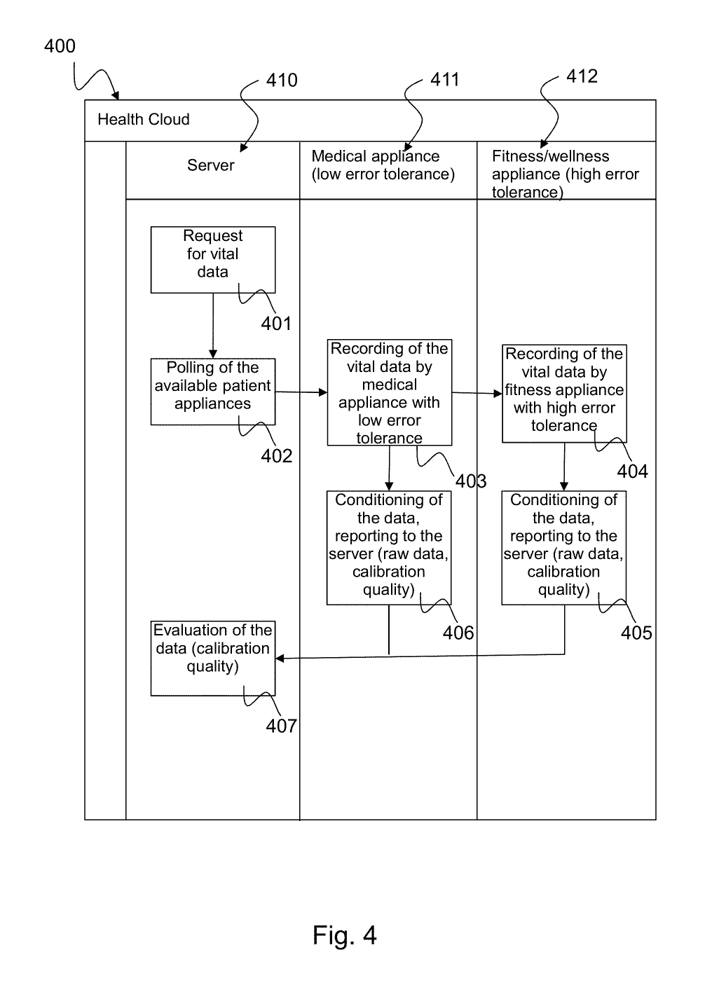

FIG. 4 shows a schematic depiction of a method 400 for data processing in a health cloud according to one embodiment.

In the detailed description that follows, reference is made to the accompanying drawings, which form a part thereof and which show, as illustration, specific embodiments in which the invention can be implemented. It goes without saying that other embodiments can also be used and structural or logical changes can be made without departing from the concept of the present invention. The detailed description that follows is therefore not intended to be understood in a restricting sense. Further, it goes without saying that the features of the different exemplary embodiments described herein can be combined with one another unless specifically indicated otherwise.

The aspects and embodiments are described with reference to the drawings, wherein like reference symbols generally denote like elements. In the description that follows, numerous specific details are set out for explanatory purposes in order to convey a detailed understanding of one or more aspects of the invention. However, it may be obvious to a person skilled in the art that one or more aspects or embodiments can be implemented with a smaller degree of the specific details. In other cases, known structures and elements are depicted in schematic form in order to facilitate the description of one or more aspects or embodiments. It goes without saying that other embodiments can be used and structural or logical changes made without departing from the concept of the present invention.

Even though a particular feature or a particular aspect of an embodiment may have been disclosed in respect of only one of multiple implementations, it is moreover possible for such a feature or such an aspect to be combined with one or more other features or aspects of other implementations, as may be desirable and advantageous for a given or particular application. In addition, insofar as the expressions “include”, “have”, “having” or other variants thereof are used either in the detailed description or in the claims, such expressions are intended to be inclusive in a manner similar to the expression “comprise”. The expressions “coupled” and “connected” may have been used together with derivations therefrom. It goes without saying that such expressions are used to indicate that two elements cooperate or interact with one another independently of whether they are in direct physical or electrical contact or are not in direct contact with one another. Moreover, the expression “by way of example” is meant to be regarded merely as an example instead of the designation for the best or optimum. The description that follows is therefore not intended to be understood in a limiting sense.

FIG. 1 shows a schematic depiction of a measurement data processing system 100 according to one embodiment. The measurement data processing system 100 comprises a measurement data server 101 and also first 103 and second 105 measurement data capture devices.

The measurement data server 101 is used for providing first measurement data 102 having a first error tolerance 110 and second measurement data 104 having a second error tolerance 112, the first error tolerance 110 being lower than the second error tolerance 112, for example it is possible for the first error tolerance to be in the region of 0.01% while the second may be in the region of 0.1%. Subsequently, the first error tolerance is referred to as the low error tolerance, while the second error tolerance is referred to as the high error tolerance.

The first measurement data capture device 103 is used for capturing the first measurement data 102. The first measurement data capture device 103 is calibrated for capturing the first measurement data 102 having a first error tolerance 110. The first measurement data capture device 103 has a first measurement data transmission interface 107 that can be used to transmit the first measurement data 102 and a first piece of calibration information 106 about the calibration of the first measurement data capture device 103 to the measurement data server 101.

The second measurement data capture device 105 is used for capturing the second measurement data 104. The second measurement data capture device 105 is calibrated for capturing the second measurement data 104 having a second error tolerance 112. The second measurement data capture device 105 has a second measurement data transmission interface 109 that can be used to transmit the second measurement data 104 and a second piece of the calibration information 108 about the calibration of the second measurement data capture device 105 to the measurement data server 101.

The measurement data server 101 has a measurement data capture interface 111 that can be used to receive the first measurement data 102 and the first calibration information 106 from the first measurement data capture device 103 and that can be used to receive the second measurement data 104 and the second calibration information 108 from the second measurement data capture device 105.

The measurement data server 101 is further used to provide the first measurement data 102 and the second measurement data 104 by pointing to the first calibration information 106 and the second calibration information 108 of the applicable measurement data capture devices 103, 105.

The first calibration information 106 can comprise a piece of information about a quality of the first measurement data 102, and the second calibration information 108 can comprise a piece of information about a quality of the second measurement data 104.

The first measurement data capture device 103 may be a medical appliance for capturing vital data having a prescribed first error tolerance 110.

The first measurement data capture device 103 can have a compliance with a medical standard, particularly a compliance pursuant to the Medical Devices Act.

The first measurement data capture device 103 can have a gauging stamp, and the first calibration information 106 may be based on the gauging stamp.

The second measurement data capture device 105 may be an appliance for capturing vital data, for example a fitness appliance, a wellness appliance, a consumer product for home use, particularly a heart monitor, a pedometer, a blood pressure gauge or a wearable appliance.

The second measurement data capture device 105 can have an identifier, particularly a serial number, that can indicate a design of the second measurement data capture device 105. The second calibration information 108 may be based on the identifier.

The measurement data server 101 can store the received first measurement data 102 in a first database and can store the received second measurement data 104 in a second database. The second database may be separate from the first database. The first measurement data 102 stored in the first database can have a pointer to the second measurement data 104 stored in the second database.

The first measurement data 102 stored in the first database may be provided with a reference that can refer to the second measurement data 104 stored in the second database.

The reference can comprise the second calibration information 108 of the second measurement data capture device 105.

The reference can comprise a form factor that can indicate a Q-factor of the second measurement data 104 in relation to a Q-factor of the first measurement data 102.

The measurement data processing system 100 can be used, by way of example, in a medical setting, e.g. in a health cloud or a decentralized system 200 for processing vital data, as described below in regard to FIG. 2. In the general form shown in FIG. 1, the measurement data processing system 100 can also be used in other areas, however, in order to base the measurement on a larger database and hence render it more informative and more accurate. By way of example, the measurement data processing system 100 can be used in an automotive setting in which calibrated measuring instruments provide data of high accuracy, for example speed sensors, pressure sensors or temperature sensors. In addition, it is possible for further measuring instruments from around the driver to be used to attain a better overview, however, for example measurement data that are recorded by mobile subscriber terminals, such as voice messages, up-to-date route data from the Internet or recorded vital data from wellness appliances to identify the fitness of the driver to drive.

In this case, the second measurement data capture device 105 affords the advantage of a higher level of flexibility. Particularly if said device does not have to have complex calibration according to legal directives, it is easier for the second measurement data capture device 105 to be replaced or loaded with new software in order to flexibly match changed environmental situations.

By way of example, the measurement data processing system 100 can also be used in a traffic control system that, by way of example, has permanently installed installations having a low error tolerance for capturing the location, speed and license plate numbers of vehicles. In addition, this traffic control system can also use information from other measured value capture systems having higher error tolerance, such as e.g. mobile appliances, for example navigation appliances in vehicles or mobile communication appliances such as smartphones, notebooks with a mobile radio adapter or other appliances with a mobile radio or Internet connection. The first measurement data 102 having the first error tolerance 110 can be recorded by such permanently installed appliances, for example, and the second measurement data 104 having the second error tolerance can be recorded by such mobile appliances.

The second measurement data 104 can also be used to check the first measurement data. Over the course of time, it may thus arise that the calibration of the first measurement data capture device 103 no longer meets the requirements. By recording the second measurement data 104 using the second measurement data capture device 105, it is possible to identify a deviation in the accuracy of the first measurement data 102 and to trigger an alarm if necessary. When the calibration Q-factor of the first measurement data 102 is known, a threshold value can be determined. If the difference between first measurement data 102 and second measurement data 104 is outside the threshold value, an error can be identified and, by way of example, an alarm can be triggered in order to have the first measurement data capture device 103 recalibrated.

FIG. 2 shows a schematic depiction of a decentralized system 200 for processing vital data according to one embodiment. The decentralized system 200 comprises one or more decentralized communication appliances 201, 203, 205 that are designed to communicate with one or more other decentralized communication appliances 201, 203, 205. For example, each of the decentralized communication appliances 201, 203, 205 can use mobile patient software that can be executed on a communication appliance that has a piece of decentralized data merging software. The decentralized communication appliances 201, 203, 205 can access and store data from subscribers in a distributed manner, for example on the subscriber communication appliance 205, without these data needing to be stored on a central server or on a plurality of distributed servers.

Each of the decentralized communication appliances 201, 203, 205 can initiate a communication with another of the decentralized communication appliances 201, 203, 205 in the decentralized network 210. It is thus possible for the decentralized network 102 to perform scaling for services and actions to be performed. For example, when a task to be performed is identified on the subscriber communication appliance 205, this appliance 205 can decide whether an identified event that signals a task to be performed needs to be escalated, so that one or more subscriber appliances 205 are made aware thereof, or whether the nursing service team needs to be informed about said event. If the subscriber communication appliance 205 decides that the nurse should be made aware, for example, then the subscriber communication appliance 205 can use functionality of the decentralized data merging software to convey an applicable event, for example an alarm or a report, directly to the nurse communication appliance 201. The nurse communication appliance 201 can then receive the report and trigger an applicable user interface event, e.g. on the screen, as a sound or as a vibration. Further, it can convey an applicable response, for example that the task to be performed is being undertaken, to the subscriber communication appliance 205.

The decentralized system 200 comprises a cloud services server that the network 210 can use to access other networks or components in the cloud. Further, the decentralized system 200 comprises diagnosis/monitoring systems 207 that can be used to perform a diagnosis or monitoring of the network 210 and of the communication appliances 201, 203, 205. The diagnosis/monitoring systems 207 can be used by doctors or nursing staff to diagnose subscriber appliances 205, monitor the effectiveness of interventional measures and the like. By way of example, the efficiency of a care plan can be monitored or it is possible for diagnosis measures and possible emergency plans to be created and checked.

The decentralized system 200 further comprises medical sensor appliances 213 for capturing vital data of the patient, control appliances 211 for controlling the measured value capture and additionally fitness/wellness appliances 215 that can likewise be used to capture vital data of the patient. The medical sensor appliances 213, the fitness/wellness appliances 215 and the control appliances 211 can be controlled by a subscriber communication appliance 205, so that any doctor/nurse can use his mobile subscriber communication appliance 205 to initiate a measured value capture on the patient.

The medical sensor appliances 213 can have a very low error tolerance and may be compliant with the Medical Devices Act, for example. They may correspond e.g. to the first measurement data capture devices 103 according to the description in regard to FIG. 1. By contrast, the fitness/wellness appliances 215 can have a high error tolerance, i.e. can perform far more inaccurate measurement of the vital data in comparison with the medical sensor appliances 213, and in general may not be compliant with the Medical Devices Act. The fitness/wellness appliances 215 can correspond e.g. to the second measurement data capture devices 105 according to the description in regard to FIG. 1.

In the network 210, a method for evaluating the data, recorded by the medical sensor appliances 213 and the fitness/wellness appliances 215, can proceed as follows, for example. The method can perform an evaluation of vital data, the vital data being recorded by appliances with error tolerance of a first kind 213, i.e. the medical sensor appliances 213, and by appliances of a second kind, i.e. the fitness/wellness appliances. In this case, the error tolerance of the first kind is lower, in particular substantially lower, than the error tolerance of the second kind. The appliances with error tolerance of the first kind can have an identifier that is stored in a database, and the respective appliance is identified on the basis of the identifier as an appliance with error tolerance of the first kind for the medical health care. The appliances with error tolerance of the second kind have substantially no identifier, and data of these appliances with error tolerance of the second kind are conditioned for the recording of vital data according to their calibration Q-factor. In this case, vital data from appliances with error tolerance of the second kind can be stored in a database for the medical health care by taking into consideration the calibration quality, for example using the diagnosis/monitoring systems 207 or using the connection to the cloud services 209.

The data of the appliances with error tolerance of the first kind can be stored in a first database, and the data of the appliances with error tolerance of the second kind can be stored in a separate second database, and data of the second database can be added to the data of the first database by means of references.

In one embodiment of the method, the reference from the first database to the second database adds information about the calibration Q-factor or the error tolerance of the first kind, particularly as a form factor.

In one embodiment of the method, the reference permits abstraction of the data such that no inference of personal data that may be included in the respective data is permitted.

A system can perform the method and authenticate the communication appliances connected to the Internet.

The method can be performed using a computer program. A computer program product can be used for the execution of the computer program to perform the method.

FIG. 3 shows a schematic depiction of a method 300 for measurement data processing according to one embodiment. The method 300 can have the following steps: 1^{st} step: capture 301 of first measurement data by a first measurement data capture device, wherein the first measurement data capture device is calibrated for capturing the first measurement data having a first error tolerance, and transmission of the first measurement data and a first piece of calibration information about the calibration of the first measurement data capture device. 2^{nd} step: capture 302 of second measurement data by a second measurement data capture device, wherein the second measurement data capture device is calibrated for capturing the second measurement data having a second error tolerance, the first error tolerance being lower than the second error tolerance, and transmission of the second measurement data and a second piece of calibration information about the calibration of the second measurement data capture device. 3^{rd} step: reception 303 of the first measurement data and the first calibration information from the first measurement data capture device, and reception of the second measurement data and the second calibration information from the second measurement data capture device. 4^{th} step: provision 304 of the first measurement data and the second measurement data by pointing to the first calibration information and the second calibration information of the applicable measurement data capture devices.

The method 300 can proceed on a measurement data processing system 100 as described in regard to FIG. 1. The first calibration information can comprise a piece of information about a quality of the first measurement data, and the second calibration information can comprise a piece of information about a quality of the second measurement data. The method 300 can further have the following step: display of the second calibration information in respect of a compliance with a medical standard, particularly in respect of a compliance pursuant to the Medical Devices Act.

The method 300 can be carried out on the decentralized system 200 described in FIG. 2 or on the measurement data processing system 100 shown in FIG. 1.

FIG. 4 shows a schematic depiction of a method 400 for data processing in a health cloud according to one embodiment.

The method 400 for data processing in a health cloud comprises the following steps according to the depiction in FIG. 4: 1^{st} step 401: request for vital data; 2^{nd} step 402: polling of the available patient appliances; 3^{rd} step 403: recording of the vital data by medical appliance with low error tolerance and with quantitative calibration, possibly gauged; 4^{th} step 404: recording of the vital data by the fitness appliance with high error tolerance and with qualitative calibration, possibly ungauged; 5^{th} step 405: conditioning of the data (of the medical appliance) and reporting of the raw data and calibration quality to the server; 6^{th} step 406: conditioning of the data (of the fitness appliance) and reporting of the raw data and calibration quality to the server; 7^{th} step 407: evaluation of the data by taking into consideration the calibration quality.

The method can be carried out in the health cloud 413, for example the decentralized system 200 for processing vital data, as described in FIG. 2. Steps 1, 2 and 7 may be implemented on a server 410 in the health cloud 413, steps 3 and 6 may be implemented on a medical appliance 411 in the health cloud and steps 4 and 5 may be implemented on a fitness appliance 412 in the health cloud. The server 410 may be a measurement data server 101, as described above in regard to FIG. 1. The medical appliance 411 may be a first measurement data capture device 103, as described above in regard to FIG. 1. The fitness appliance 412 may be a second measurement data capture device 105, as described above in regard to FIG. 1. The medical appliance 411 has a low error tolerance in comparison with the fitness appliance 412. Therefore, the medical appliance 411 is also referred to as an appliance of a first kind or of a first type that is specified by its low error tolerance. The fitness appliance 412 is referred to as an appliance of a second kind or of a second type that is specified by its high error tolerance in comparison with that of the medical appliance. By way of example, the error tolerance of the medical appliance 411 may be in the region of 0.01%, while the error tolerance of the fitness appliance 412 may be in the region of 0.1%. These are merely examples, and other numerical values involving the error tolerance of the medical appliance 411 being lower or much lower than the error tolerance of the fitness appliance 412 are equally possible.

The method 400 relates to the data processing in a health cloud 413. The health cloud 413 is a data processing system for processing data from substantially medical appliances that meet special demands on medical data recording and data processing. The data processing system can also be opened on a case-by-case basis, using this method 400, for data from the sphere of wearable appliances, including wearables (i.e. portable appliances) that come from the realm of consumer products that are available to the patient for home use.

On a case-by-case basis means that the data from appliances of the patient allow information about the course and occurrence of an illness at a stage at a time when the patient was not yet under the medical treatment of a doctor or healer.

Should the doctor or healer be of the opinion that appliances of the patient deliver important information about vital parameters, such as pulse, blood pressure, breathing, for example, for diagnosing an illness, such as a heart attack, for example, then the method 400 allows him to be able to qualitatively add data to the system 413 by taking into consideration the low error tolerance.

In this case, the method proceeds substantially as follows: the health cloud 413 comprises a server 410 on which data in the first category are stored. The health cloud 413 has primarily medical appliances 411 of low error tolerance recorded in it. The low error tolerance of the appliances 411 is stipulated primarily by the Medical Devices Act.

In addition, there are fitness appliances 412, such as heart rate monitors or pedometers, for example, that have a high level of error tolerance and are not subject to the Medical Devices Act. These can be recorded in the health cloud 413 on a case-by-case basis, particularly if, in a medical emergency, the data of the heart rate monitors of other fitness appliances make the evaluation of the data appear desirable.

The fitness appliance 413 can be connected to the health cloud 413 particularly via a mobile communication interface, for example Bluetooth. The patient or the healer who incorporates the appliance 412 can therefore be immediately provided with a pointer to the calibration quality and the compliance with the standard of the Medical Devices Act. This can be accomplished by means of signaling in traffic light colors, for example, with a low deviation being able to be denoted by a green signal color in the measured value display and a higher deviation being able to be displayed by a yellow or red signal color.

The method 400 can specifically proceed as follows:

in step 401, vital data are called for only from the different kinds of appliance.

In step 402, the available patient appliances are polled.

In step 403, the vital data are recorded by the medical appliance 411 with low error tolerance and with qualitative calibration, said medical appliance possibly also being gauged.

In step 104, the vital data are recorded by the fitness appliance 412 with high error tolerance and with qualitative calibration, said fitness appliance being substantially ungauged, by means of a reference.

In step 405, the data are conditioned and the raw data and calibration quality are reported to the server 410.

In step 406, the data are conditioned and the raw data and calibration quality are reported to the server 410. The conditioning can be accomplished by adding a form factor.

In step 407, the data are evaluated by taking into consideration the calibration quality.

The appliance data and calibration quality can also be stored in a database on the basis of the design compliance. The appliances can then be identified in a trust center on the basis of the identifier, particularly a serial number. This renders the computation of the error tolerance obsolete, and the manufacturer details can be used preferably for ascertaining the calibration tolerance.

The method 400 can therefore be used to incorporate appliances 412 of high error tolerance into a medical data capture system (the health cloud 413) having appliances 411 of substantially low error tolerance on a case-by-case basis, identical measured variables from different systems also being able to be captured on the basis of multisensory modules. It is thus possible for the appliances 412, 413 to communicate in automated fashion and to allow the doctor or nursing staff a more accurate analysis of the state of the patient on the basis of the larger measurement database.

It is therefore possible for the diagnosis of the patient to be based on an extensive information base, i.e. it is possible not only for the data of the medical appliances 411 alone to be used for the diagnosis, but also for measurement data produced by patients from the private setting to be included in the diagnosis as well.

The method 400 can be carried out on the decentralized system 200 described in FIG. 2 or on the measurement data processing system 100 shown in FIG. 1.

One aspect of the invention also comprises a computer program product that can be loaded directly into the internal memory of a digital computer and comprises software code sections that can be used to carry out the method 200, 400 described in regard to FIGS. 2 and 4 when the product runs on a computer. The computer program product may be stored on a computer-compatible medium and comprise the following: computer-readable program means that prompt a computer to capture first measurement data by means of a first measurement data capture device, wherein the first measurement data capture device is calibrated for capturing the first measurement data having a first error tolerance and to transmit the first measurement data and a first piece of calibration information about the calibration of the first measurement data capture device; to capture second measurement data by means of a second measurement data capture device, wherein the second measurement data capture device is calibrated for capturing the second measurement data having a second error tolerance, the first error tolerance being lower than the second error tolerance, and to transmit the second measurement data and a second piece of calibration information about the calibration of the second measurement data capture device; to receive the first measurement data and the first calibration information from the first measurement data capture device and to receive the second measurement data and the second calibration information from the second measurement data capture device; and to provide the first measurement data and the second measurement data by pointing to the first calibration information and the second calibration information of the applicable measurement data capture devices.

The computer may be a PC, for example a PC in a computer network. The computer may be realized as a chip, an ASIC, a microprocessor or a signal processor and be arranged in a computer network, for example a measurement data processing system 100 as described in FIG. 1 or in a decentralized system as described in FIG. 2 or in a cloud.

It goes without saying that the features of the different embodiments described by way of example herein can be combined with one another unless specifically indicated otherwise. As depicted in the description and the drawings, single elements that have been depicted as being connected do not have to be directly connected to one another; intermediate elements may be provided between the connected elements. Further, it goes without saying that embodiments of the invention can be implemented in single circuits, partly integrated circuits or completely integrated circuits or programming means. The term “by way of example” is meant merely as an example and not as the best or optimum. Particular embodiments have been illustrated and described herein, but it is obvious to a person skilled in the art that a multiplicity of alternative and/or homogeneous implementations can be realized instead of the embodiments shown and described, without deviating from the concept of the present invention.

### LIST OF REFERENCE SYMBOLS

100: measurement data processing system according to one embodiment
101: measurement data server
102: first measurement data
103: first measurement data capture device
104: second measurement data
105: second measurement data capture device
106: first calibration information
107: first measurement data transmission interface
108: second calibration information
109: second measurement data transmission interface
110: first error tolerance
111: measurement data capture interface
112: second error tolerance
121: first measurement
122: second measurement
200: decentralized system for processing vital data
201: module for nurse
203: support network
205: module for subscribers in the system
207: diagnosis/monitoring systems
209: cloud services
210: network
211: control appliances
213: medical sensor appliances
215: fitness/wellness appliances
300: method for measurement data processing according to one embodiment
301: 1^{st} step: capture of first measurement data
302: 2^{nd} step: capture of second measurement data
303: 3^{rd} step: reception of the first/second measurement data
304: 4^{th} step: provision of the first/second measurement data
400: method for data processing in a health cloud according to one embodiment
401: 1^{st} step: request for vital data
402: 2^{nd} step: polling of the available patient appliances
403: 3^{rd} step: recording of the vital data by medical appliance with low error tolerance
404: 4^{th} step: recording of the vital data by fitness appliance with high error tolerance
405: 5^{th} step: conditioning of the data (of the medical appliance), reporting to the server
406: 6^{th} step: conditioning of the data (of the fitness appliance), reporting to the server
407: 7^{th} step: evaluation of the data

1.-15. (canceled)

16. A measurement data processing system, comprising:
a measurement data server for providing first measurement data having a first error tolerance and second measurement data having a second error tolerance, the first error tolerance being lower than the second error tolerance;
a first measurement data capture device for capturing the first measurement data, wherein the first measurement data capture device is calibrated for capturing the first measurement data having a first error tolerance and wherein the first measurement data capture device has a first measurement data transmission interface that is configured to transmit the first measurement data and a first piece of calibration information about the calibration of the first measurement data capture device to the measurement data server; and
a second measurement data capture device for capturing the second measurement data, wherein the second measurement data capture device is calibrated for capturing the second measurement data having a second error tolerance and wherein the second measurement data capture device has a second measurement data transmission interface that is configured to transmit the second measurement data and a second piece of calibration information about the calibration of the second measurement data capture device to the measurement data server,
wherein the measurement data server has a measurement data capture interface that is configured to receive the first measurement data and the first calibration information from the first measurement data capture device and to receive the second measurement data and the second calibration information from the second measurement data capture device,
wherein the measurement data server is further configured to provide the first measurement data and the second measurement data by pointing to the first calibration information and the second calibration information of the applicable measurement data capture devices.

17. The measurement data processing system as claimed in claim 16,
wherein the first calibration information comprises a piece of information about a quality of the first measurement data and wherein the second calibration information comprises a piece of information about a quality of the second measurement data.

18. The measurement data processing system as claimed in claim 16,
wherein the first measurement data capture device is a medical appliance for capturing vital data having a prescribed first error tolerance.

19. The measurement data processing system as claimed in claim 16,
wherein the first measurement data capture device has a compliance with a medical standard, particularly a compliance pursuant to the Medical Devices Act.

20. The measurement data processing system as claimed in claim 16,
wherein the first measurement data capture device has a gauging stamp and the first calibration information is based on the gauging stamp.

21. The measurement data processing system as claimed in claim 16,
wherein the second measurement data capture device is one of the following appliances for capturing vital data: a fitness appliance, a wellness appliance, a consumer product for home use, particularly a heart rate monitor, a pedometer, a blood pressure gauge or a wearable appliance.

22. The measurement data processing system as claimed in claim 16,
wherein the second measurement data capture device has an identifier, particularly a serial number, that indicates a design of the second measurement data capture device, the second calibration information being based on the identifier.

23. The measurement data processing system as claimed in claim 16,
wherein the measurement data server is designed to store the received first measurement data in a first database and to store the received second measurement data in a second database, wherein the second database is separate from the first database and wherein the first measurement data stored in the first database have a pointer to the second measurement data stored in the second database.

24. The measurement data processing system as claimed in claim 23,
wherein the first measurement data stored in the first database are provided with a reference that refers to the second measurement data stored in the second database.

25. The measurement data processing system as claimed in claim 24,
wherein the reference comprises the second calibration information of the second measurement data capture device.

26. The measurement data processing system as claimed in claim 25,
wherein the reference comprises a form factor that indicates a Q-factor of the second measurement data in relation to a Q-factor of the first measurement data.

27. A method for measurement data processing, having the following steps:
capturing first measurement data by a first measurement data capture device, wherein the first measurement data capture device is calibrated for capturing the first measurement data having a first error tolerance, and transmission of the first measurement data and a first piece of calibration information about the calibration of the first measurement data capture device;
capturing second measurement data by a second measurement data capture device, wherein the second measurement data capture device is calibrated for capturing the second measurement data having a second error tolerance, the first error tolerance being lower than the second error tolerance, and transmission of the second measurement data and a second piece of calibration information about the calibration of the second measurement data capture device;
receiving the first measurement data and the first calibration information from the first measurement data capture device, and reception of the second measurement data and the second calibration information from the second measurement data capture device; and
providing the first measurement data and the second measurement data by pointing to the first calibration information and the second calibration information of the applicable measurement data capture devices.

28. The method as claimed in claim 27,
wherein the first calibration information comprises a piece of information about a quality of the first measurement data and wherein the second calibration information comprises a piece of information about a quality of the second measurement data.

29. The method as claimed in claim 27, further comprising:
displaying the second calibration information in respect of a compliance with a medical standard, particularly in respect of a compliance pursuant to the Medical Devices Act.

30. A computer program having a program code for performing the method as claimed in claim 27 when the computer program is executed on a computer.
